(19) United States
(12) Patent Application Publication
(10) Pub. No.: US 2013/0123433 A1
(43) Pub. Date: May 16, 2013

(54) METHOD FOR PREPARING POLY(ALLYLAMINE) HYDROCHLORIDE AND DERIVATIVES THEREFROM

(71) Applicants: YU-LIANG LIU, TAOYUAN (TW); CHING-PENG WEI, TAOYUAN (TW)

(72) Inventors: YU-LIANG LIU, TAOYUAN (TW); CHING-PENG WEI, TAOYUAN (TW)

(73) Assignee: FORMOSA LABORATORIES, INC., TAOYUAN (TW)

(21) Appl. No.: 13/295,528

(22) Filed: Nov. 14, 2011

(51) Int. Cl. C08F 126/02; B01D 63/00; A61P 3/00

(52) U.S. Cl. 525/328.2; 526/310; 210/321.6; 264/239; 264/310; 264/140

(57) ABSTRACT

The present invention provides a method for preparing poly(allylamine) hydrochloride, sevelamer hydrochloride, sevelamer carbonate and colesevelam hydrochloride. The present invention also relates to a process for preparing oly(allylamine) hydrochloride, sevelamer hydrochloride, sevelamer carbonate and colesevelam hydrochloride with low allylamine content and high specific gravity.

### FIELD OF THE INVENTION

The present invention relates to a method for preparing poly(allylamine) hydrochloride, sevelamer hydrochloride, sevelamer carbonate and colesevelam hydrochloride. The present invention also relates to a process for preparing poly(allylamine) hydrochloride, sevelamer hydrochloride, sevelamer carbonate and colesevelam hydrochloride with low allylamine content and high specific gravity.

### DESCRIPTION OF PRIOR ART

Poly(allylamine) hydrochloride is a cationic polyelectrolyte prepared by the polymerization of allylamine. It can be used in combination with an anionic polyelectrolyte like poly(sodium styrene sulfonate) to form a layer-by-layer adsorbed film of negatively and positively charged polymers. Poly(allylamine) hydrochloride has many biomedical applications.

Sevelamer is a phosphate binding drug used to prevent hyperphosphatemia in patients with chronic renal failure. When taken with meals, sevelamer binds to dietary phosphate and prevents its absorption. Sevelamer is a copolymer of 2-(chloromethyl)oxirane (epichlorohydrin) and prop-2-en-1-amine. The amine groups of sevelamer become partially protonated in the intestine and interact with phosphorus molecules through ionic and hydrogen bonding. Sevelamer hydrochloride is poly(allylamine) hydrochloride crosslinked with epichlorohydrin in which 40% of the amines are protonated. It is known chemically as poly(allylamine-co-N,N′-diallyl-1,3-diamino-2-hydroxypropane) hydrochloride. Sevelamer hydrochloride is hydrophilic, but insoluble in water. The marketed form sevelamer hydrochloride is a partial hydrochloride salt being present as approximately 40% amine hydrochloride and 60% sevelamer base. Sevelamer carbonate) is known chemically as poly(allylamine-co-N,N′-diallyl-1,3-diamino-2-hydroxypropane) carbonate salt. Sevelamer carbonate is hygroscopic, but insoluble in water. Sevelamer carbonate, which contains the same active moiety as sevelamer carbonate hydrochloride

Colesevelam hydrochloride is a cholesterol-lowering agent that has been shown to lower LDL cholesterol. Colesevelam hydrochloride has been described in U.S. Pat. Nos. 5,607,669, 5,624,963, 5,679,717, 5,693,675, 5,917,007, and 5,919,832.

Phosphate-binding polymers are publicly known as cationic polymer compounds comprising primary and secondary amines which are prepared by crosslinking polyallyamine with the use of a crosslinking agent such as epichlorohydrin. Phosphate-binding polymers include sevelamer hydrochloride, which is marketed under the brand name RenaGel® (Genzyme), and polymers prepared using the methods disclosed in U.S. Pat. No. 5,667,775. U.S. Pat. No. 7,014,846 discloses phosphate-binding polymers that are provided for removing phosphate from the gastrointestinal tract and used for oral administration.

Methods to produce poly(allylamine) hydrochloride are known. For example, Dimitri R. et al., J. Applied Polymer Science, Vol. 80, 2073-2083 (2001) describes the poly(allylamine) hydrochloride (PAA-HCl) hydrogels that are synthesized by chemically crosslinking with epichlorohydrin (ECH). U.S. Pat. Nos. 6,180,754 B1, 6,362,266 and PCT Publication No. WO01/180721 A1 describes a process for producing a cross-linked poly-allylamine polymer. U.S. Pat. No. 5,496,545 discloses a method of purifying poly(allylamine) hydrochloride (PAA-HCl) from a crude PAA-HCl reaction mixture by a precipitation reaction in methanol solution. It was observed that low molecular weight poly(allylamine) oligomers could dissolve into the methanol, but high molecular weight polymers does not dissolve in it. The solid PAA-HCl (precipitate material) formed in a chemical reaction can be separated by filtration and reiterated above-mentioned procedure for the precipitation with methanol. The process is repeated at least three times for each batch and large amounts of organic solvent (about 15-20 fold of sample volume) required to cause precipitation. It is obvious that the process for producing good quality of PAA-HCl is very laborious, not cost-effective and also not a environment-friendly.

The existing methods to produce poly(allylamine) hydrochloride create difficult and costly handling and cleanup procedures, especially in an industrial production process. The present invention addresses these problems and gives a more practical solution to them.

### SUMMARY OF THE INVENTION

The present invention provides a membrane filtration system for separating polymeric liquid with different molecular weights comprising:
(a) one or more membrane filtration units, wherein the membrane filtration unit compiled in a pressure vessel;
(b) a feeding pipe coupled to the membrane filtration unit, wherein the feeding pipe with one end fixed at one end of the pressure vessel;
(c) a concentrate exit pipe coupled to the membrane filtration unit, wherein the concentrate exit pipe with one end fixed at one end of the pressure vessel, and the other end of the concentrate exit pipe coupled to the feeding pipe by a succeeding unit;
(d) a filtrate tank surrounded by the membrane filtration unit(s), which for collecting a filtrate liquid that flows from the pressure vessel;
(e) a filtrate exit pipe connected to said filtrate tank, which involves removing the filtrate liquid from the filtrate tank; and
(f) a pump system coupled to the membrane filtration unit, which provides a pressure and forces a feed liquid against the membrane filtration unit.

The present invention provides a method for preparing poly(allylamine) hydrochloride comprising the steps of:
(a) reacting an aqueous of allylamine hydrochloride with a polymerization agent to produce a crude poly(allylamine) hydrochloride; and
(b) performing the crude poly(allylamine) hydrochloride to prepare poly(allylamine) hydrochloride with an average molecular weight of over 2,000 daltons by using a membrane filtration system.

The present invention also provides a process for producing a dried sevelamer hydrochloride comprising the steps of:
(a) providing a wet sevelamer hydrochloride;
(b) performing the washing procedure with water to achieve good quality of wet sevelamer hydrochloride;
(c) compressing the wet sevelamer hydrochloride with compressing process; and
(d) drying the compressed sevelamer hydrochloride.

The present invention further provides a process for producing a dried sevelamer carbonate comprising the steps of:
(a) providing a crosslinked poly(allylamine) hydrochloride;
(b) neutralizing the crosslinked poly(allylamine) polymer with metal carbonate or carbon dioxide to produce a wet sevelamer carbonate;
(c) performing the washing procedure with water to achieve good quality of wet sevelamer carbonate;
(d) compressing the wet sevelamer carbonate with compressing process; and
(e) drying the compressed sevelamer carbonate.

The present invention also relates to a process for producing a dried colesevelam hydrochloride comprising the steps of:
(a) providing a crosslinked poly(allylamine) hydrochloride;
(b) neutralizing the crosslinked poly(allylamine) hydrochloride with an alkaline solution;
(c) alkylating the crosslinked poly(allylamine) polymer with 1-bromodecane and (6-bromohexyl)trimethylammonium bromide (BHTAB) to produce a crude colesevelam;
(d) reacting the crude colesevelam with sodium chloride;
(e) applying the mixture from step (d) with anion exchange to produce wet colesevelam hydrochloride;
(f) performing the washing procedure with water to achieve good quality of wet colesevelam hydrochloride; and
(g) drying the wet colesevelam hydrochloride.

### DETAILED DESCRIPTION OF THE INVENTION

The present invention provides a membrane filtration system for separating polymeric liquid with different molecular weights comprising:
(a) one or more membrane filtration units, wherein the membrane filtration unit compiled in a pressure vessel;
(b) a feeding pipe coupled to the membrane filtration unit, wherein the feeding pipe with one end fixed at one end of the pressure vessel;
(c) a concentrate exit pipe coupled to the membrane filtration unit, wherein the concentrate exit pipe with one end fixed at one end of the pressure vessel, and the other end of the concentrate exit pipe coupled to the feeding pipe by a succeeding unit;
(d) a filtrate tank surrounded by the membrane filtration unit(s), which for collecting a filtrate liquid that flows from the pressure vessel;
(e) a filtrate exit pipe connected to said filtrate tank, which involves removing the filtrate liquid from the filtrate tank; and
(f) a pump system coupled to the membrane filtration unit, which provides a pressure and forces a feed liquid against the membrane filtration unit.

In the present invention, the membrane filtration unit containing two or more membrane filter elements and each membrane filter element is a semi-permeable membrane having a nominal pore size.

In a preferred embodiment, the filtrate liquid is polymeric liquid with a low average molecular weight.

In a preferred embodiment, the membrane filtration system is a continuous system, which the pump system enables the membrane filtration system in a continuous motion by directing a concentrate liquid back to the feeding pipe. In the present invention, the membrane filtration system is a continuous system where the PAA-HCl aqueous solution can be added into constantly without interrupting the purification process. It is needless for size-excluding method to stop the whole purification system while comparing to using aforementioned organic solvent, which the crude PAA-HCl can not be added into the organic solvent until a purification cycle is done. Because of the continuous membrane filtration system, persons skilled in the art can easily use parallel and/or series connection of different membranes with various pore sizes to collect desired polymers. The continuous system can be supplied in automation system and useful to perform specific industrial or manufacturing processes such as producing poly(allylamine) hydrochloride and its derivatives, including sevelamer hydrochloride, sevelamer carbonate and colesevelam hydrochloride.

The present invention provides a method for preparing poly(allylamine) hydrochloride comprising the steps of:
(a) reacting an aqueous of allylamine hydrochloride with a polymerization agent to produce a crude poly(allylamine) hydrochloride; and
(b) performing the crude poly(allylamine) hydrochloride to prepare poly(allylamine) hydrochloride with an average molecular weight of over 2,000 daltons by using a membrane filtration system.

In a preferred embodiment, the membrane filtration system can filter out low molecular weight oligomers and retains high molecular weight polymers in the reaction tank. The method of the present invention only takes 4-6 hours to purify the poly(allylamine) derivatives, and the solvent used here is water. The membrane filtration system can filter out oligomers with various molecular weights according to the pore size of the membrane in this system. In an embodiment, the average molecular weight of poly(allylamine) hydrochloride ranges from 2,000 to 50,000 daltons, preferably from 10,000 to 30,000 daltons.

In the present invention, the initiator for polymerization of allylamine includes, but is not limited to 2,2′-azobis(2-methylpropionamidine)hydrochloride (AAPH).

Prior art as U.S. Pat. No. 5,496,545 shows a method for removing low molecular weight poly(allylamine) oligomers by adding crude sample (poly(allylamine) hydrochloride (PAA-HCl) aqueous solution) to a 15-20 fold volume of methanol, and causing the product to viscous and hygroscopic precipitate which is difficult to filter or centrifuge. However, using organic solvent to remove the unwanted low molecular impurities and to reach the desired purity level usually takes several days to complete a standard process, and large amount of organic solvent are used in the purification process. The organic solvent is often toxic, volatile and expensive, while the membrane filtration system used in present invention can be operated very easily, cleaned and reused many times. Thus, compared to present invention, the method described in U.S. Pat. No. 5,496,545 is not only time consuming, but also costly and is harmful to the environment.

To avoid allyamine contamination within the final products, the crude poly(allylamine) hydrochloride can be neutralized and distilled before the crosslinking reaction with ECH to give the hydrogels of polymer. The content of allylamine is not detected or less than 100 ppm in poly(allylamine) hydrochloride.

The present invention also provides a process for producing a dried sevelamer hydrochloride comprising the steps of:
(a) providing a wet sevelamer hydrochloride;
(b) performing the washing procedure with water to achieve good quality of wet sevelamer hydrochloride;
(c) compressing the wet sevelamer hydrochloride with compressing process; and
(d) drying the compressed sevelamer hydrochloride.

In the preparation of sevelamer hydrochloride, the wet sevelamer hydrochloride is prepared from reacting poly(allylamine) hydrochloride with a crosslinking agent in an alkaline solution. The crosslinking agent including, but not limited to epichlorohydrin (ECH), 1,4 butanedioldiglycidyl ether, 1,2 ethanedioldiglycidyl ether, 1,3-dichloropropane, 1,2-dichloro ethane, 1,3-dibromopropane, 1,2-dibromoethane, succinyl dichloride, dimethylsuccinate, toluene diisocyanate, acryloyl chloride, or pyromellitic dianhydride. In a preferable embodiment, the crosslinking agent is epichlorohydrin (ECH).

In a preferred embodiment, the poly(allylamine) hydrochloride is prepared by a membrane filtration system from the crude poly(allylamine) hydrochloride. In a further preferred embodiment, the crude poly(allylamine) hydrochloride is performed by neutralization and distillation before the crosslinking reaction with ECH to give the hydrogels of polymer.

In a preferred embodiment, the sevelamer hydrochloride is compressed by a compressing process, wherein the compressing process can be achieved by any ways comprising pressing, extruding, or rolling. In the preferable embodiment of present invention, the compressing process is done by a roller compactor. Through the compressing process, the specific gravity of sevelamer hydrochloride can be up to 1.22 g/cm³, and preferably greater than 1.240 g/cm³, depending on how strong compression force apply.

A drying and a milling process are further applied to the compressed sevelamer hydrochloride. The drying and milling process can be done by any methods familiar with a person skilled in the art. In a preferable embodiment of present invention, the drying machines used are tray dryer, cone Dryer, fluidized-bed dryer, Nauta dryer and granulator equipped with chopper, and milling machines used are Fitz-mill, Turbo-mill, Pin-mill and Jet-mill, respectively.

The present invention also provides a process for producing a dried sevelamer carbonate comprising the steps of:
(a) providing a crosslinked poly(allylamine) hydrochloride;
(b) neutralizing the crosslinked poly(allylamine) hydrochloride with metal carbonate or carbon dioxide to produce a wet sevelamer carbonate;
(c) performing the washing procedure with water to achieve good quality of wet sevelamer carbonate;
(d) compressing the wet sevelamer carbonate with compressing process; and
(e) drying the compressed sevelamer carbonate.

In a preferred embodiment, the crosslinked poly(allylamine) hydrochloride is prepared from reacting the poly(allylamine) hydrochloride with a crosslinking agent in an alkaline solution. The poly(allylamine) hydrochloride is prepared from a crude poly(allylamine) hydrochloride by using a membrane filtration system. The poly(allylamine) hydrochloride is performed by neutralization and distillation before the crosslinking reaction with ECH to give the hydrogels of polymer. In a preferred embodiment, the crude poly(allylamine) hydrochloride is performed with a neutralizing process by adding potassium carbonate and then disposing it for anion exchange.

In the process of the present invention, the sevelamer carbonate is compressed by a compressing process, wherein the compressing process can be achieved by any ways comprising pressing, extruding, or rolling. In a preferred embodiment, the compressing process is done by a roller compactor. Through the compressing process, the specific gravity of the sevelamer carbonate is at least 1.22 g/cm³, and preferably greater than 1.240 g/cm³. The method of the present invention further comprises milling the dried sevelamer carbonate to produce sevelamer carbonate powder.

The present invention also provides a process for producing a dried colesevelam hydrochloride comprising the steps of:
(a) providing a crosslinked poly(allylamine) hydrochloride;
(b) neutralizing the crosslinked poly(allylamine) hydrochloride with an alkaline solution;
(c) alkylating the crosslinked poly(allylamine) polymer with 1-bromodecane and (6-bromohexyl)trimethylammonium bromide (BHTAB) to produce a crude colesevelam;
(d) reacting the crude colesevelam with sodium chloride;
(e) applying the mixture from step (d) with anion exchange to produce wet colesevelam hydrochloride;
(f) performing the washing procedure with water to achieve good quality of wet colesevelam hydrochloride; and
(g) drying the wet colesevelam hydrochloride.

In a preferred embodiment, the crosslinked poly(allylamine) hydrochloride is prepared from reacting poly(allylamine) hydrochloride with a crosslinking agent in an alkaline solution. The poly(allylamine) hydrochloride is prepared from a crude poly(allylamine) hydrochloride by using a membrane filtration system. In a further preferred embodiment, the crude poly(allylamine) hydrochloride is performed by neutralization and distillation before the crosslinking reaction with ECH to give the hydrogels of polymer.

In a preferred embodiment, the method of the present invention further comprises milling the dried colesevelam hydrochloride to produce colesevelam hydrochloride powder, which the specific gravity of the colesevelam hydrochloride is at least 1.22 g/cm³, and preferably greater than 1.240 g/cm³.

Accordingly, the allylamine is no longer present (or not detected) or less than 1 ppm in the final products of sevelamer hydrochloride, sevelamer carbonate and colesevelam hydrochloride.

The term “compressing” used herein refers to any ways which can compact the gel-like product. The compressing method comprises pressing, extruding, or rolling, etc.

The term “membrane filtration” used herein refers to a technique which is used to separate materials from a liquid for the purpose of purifying it. The method used to separate molecules in solution on the basis of their size (more correctly, their hydrodynamic volume). It was usually applied to separate large molecules or macromolecular complexes such as proteins and industrial polymers. A number of different types of systems are available which specialize in filtration products, along with replacement membranes and other parts and equipment. In a membrane filtration system, a solvent is passed through a semi-permeable membrane. The membrane's permeability is determined by the size of the pores in the membrane, and it will act as a barrier to materials which are larger than the pores, while the rest of the solvent can pass freely through the membrane. There are several ways available to do size exclusion which include, but are not limited to use chromatography and ultrafiltration. Nanofiltration, ultrafiltration, microfiltration, and reverse osmosis are all membrane filtration techniques. In all cases, the size of the pores has to be carefully calculated to exclude undesirable materials, and the size of the membrane has to be designed for optimal operating efficiency. In a preferable embodiment of the present invention, a membrane filtration system is used.

This invention will be better understood by reference to the Experimental Details which follow, but those skilled in the art will readily appreciate that the specific experiments detailed are only illustrative of the invention as described more fully in the claims which follow thereafter.

### EXAMPLE

The examples below are non-limiting and are merely representative of various aspects and features of the present invention.

### Example 1

### Preparation of Poly(Allylamine) Hydrochloride

Allylamine (120 kg), 32% HCl (252 kg) and 2,2′-azobis(2-methylpropionamidine)hydrochloride (AAPH) solution (2.41 kg of AAPH was diluted with 5.4 kg of water) were prepared. Allylamine was neutralized by 32% HCl. First, HCl (32%, 252 kg) was added to a reactor and the temperature was cooled under 10° C. Allyamine (120 kg) was added in and the mixture and stirred under 40° C. by charge pump. The mixture was distilled under a vacuum below the pressure of 30 torr. The concentrated allylamine hydrochloride aqueous solution was kept and stirred in a reactor. AAPH solution was added into the reaction mixture and stirred at 49-52° C. for about 24 hours. Another portion of AAPH solution was added again and stirred for another 20˜24 hours. Then, the resulting mixture was diluted with 48 kg of water and gently mixed at room temperature. The mixture which is poly(allylamine) hydrochloride (349.4 kg) was poured into the high density polyethylene (HDPE) barrel. Water (610 kg) was added into a reactor and then the poly(allylamine) hydrochloride (115 kg) synthesized above was added in as well. The resulting poly(allylamine) hydrochloride was concentrated by a membrane filtration system to remove the impurities with an average molecular weight less than 10,000 daltons. The flow rate on the concentration process was 3.0 m³/hr. The samples were taken for analysis of viscosity.

### Example 2

### Preparation of Sevelamer Hydrochloride

Preparing PAA-HCl (59.75 g), NaOH (14.81 g), and ECH (epichlorohydrin) (2.21 g). PAA-HCl (59.75 g) and NaOH (14.81 g) were added to a beaker. The reaction mixture was cooled to 25° C. Subsequently, ECH (2.21 g) was added and a gel-like PAA-HCl crosslinked polymer (sevelamer hydrochloride) was formed in about 35 minutes later. The hydrogel of crosslinked polymer was kept stirred at room temperature for about 16 hours. Then, the gel was slurrying and washed many times with water and filtered to obtain wet cake of sevelamer hydrochloride (Table 1).

TABLE 1

| wash | solvent | weight | cake weight | filtrate weight | pH value | volume |
|---|---|---|---|---|---|---|
| 1 | PW | 176 | 104 | 140 | 9.45 | 250 |
| 2 | PW | 150 | 128 | 123 | 9.07 | 250 |
| 3 | PW | 142 | 156 | 99 | 8.67 | 250 |
| 4 | PW | 101 | 174 | 83 | 8.35 | 250 |
| 5 | PW | 116 | 193 | 94 | 7.94 | 300 |
| 6 | PW | 115 | 210 | 95 | 7.5 | 300 |
| 7 | PW | 107 | 220 | 102 | 7.04 | 300 |
| 8 | PW | 90 | 224 | 84 | 6.86 | 300 |
| 9 | PW | 87 | 223 | 85 | 6.8 | 300 |

PW pure water.

After the washing procedure, the wet cake of sevelamer hydrochloride was compressed with a roller compactor machine. The compacted sevelamer hydrochloride was then dried and milled to obtain sevelamer hydrochloride powder, which the specific gravity of sevelamer hydrochloride is at least more than 1.22 g/cm³.

### Example 3

### Preparation of Sevelamer Carbonate

Preparing PAA-HCl (450 g), NaOH (111.65 g), and epichlorohydrin (ECH, 16.63 g). PAA-HCl (450 g) and NaOH (111.65 g) were transferred to a beaker. The reaction mass was cooled to 25° C. Subsequently, ECH (16.63 g) was added and a gel-like crosslinked PAA-HCl polymer was formed after 25 min. The hydrogel of crosslinked polymer was kept stirred at room temperature for about 16 hours. K₂CO₃ (75.84 g/900 mL) was mixed with the hydrogel to proceed the anion exchange reaction. The solid was stirred under 40-45° C. for 1.5 hours and filtered to get the crude sevelamer carbonate. Finally, the hydrogel of polymer was washed with water at 40-45° C. and repeated about 9-10 times to remove unwanted impurities. The wet cake was compressed with a roller compactor machine. The compacted hydrogel of polymer was dried, milled, and to yield white to off-white sevelamer carbonate powder.

### Example 4

### Preparation of Colesevelam Hydrochloride

MeOH (250 mL) and NaOH (7.38 g), 5.1 g of PAA-HCl crosslinked polymer were added to a reaction flask equipped with a mechanical stirrer and then (6-bromohexyl)trimethylammonium bromide (BHTAB) (30 g) was added. After the reaction mixture was stirred about 20-30 min, (ECH; 6%) was added to the mixture. The reaction mixture was heated to reflux at 70° C. for about 18 hours. The reaction was cooled to room temperature, and filtered off to get 144 g of wet cake. The cake was washed with 200 g of MeOH for three times for 15 min each, then washed with 200 mL of 11% NaCl aqueous solution three times for 15 min each, and then washed by slurrying in 200 mL water three times for 15 min each and filtered off to get 159 g wet cake. Transferred the wet cake to the beaker and then added 200 mL of water and 6.5 g of 32% HCl to adjust the pH to 4˜5. Filtered off and to get about 181 g wet cake. The cake was washed with 200 g of water twice for 10 min each. Finally, the wet cake was washed with 200 g of MeOH twice for 10 min each and filtered off to get 219 g wet cake of colesevelam hydrochloride. The cake was dried, milled and the final colesevelam hydrochloride product was obtained.

### Example 5

### Preparation of Poly(Allylamine) Hydrochloride with not-Detectable or Less Than 100 ppm of Allylamine

40% PAA-HCl (133.8 g), 45% NaOH (33.2 g) was added to a reaction flask. The mixture was heated to 110° C. for azeotropic distillation to remove unreacted allylamine. After heating, the distillate was obtained about 24 g. The small sample was taken to determine the allylamine content, which is less than 100 ppm in its final product of poly(allylamine) hydrochloride.

### Example 6

### Preparation of Sevelamer Hydrochloride with not-Detectable or Less than 1 ppm of Allylamine

40% PAA-HCl (133.8 g), 45% NaOH (33.2 g) was added to a reaction flask. The mixture was heated to 110° C. for azeotropic distillation to remove unreacted allylamine. After heating, the distillate was obtained about 24 g. The small sample was taken to determine the allylamine content, which is not more than 100 ppm in its final product of poly(allylamine) hydrochloride. When the reaction meets the requirements of IPC criteria, added 42 g water and 4.5 g epichlorohydrin (ECH) to perform the crosslinking reaction, a gel-like crosslinked PAA-HCl polymer (sevelamer hydrochloride) was formed in about 25 minutes later. The gel was washed many times with water till meet the requirements of IPC criteria. The resulting cake was dried and milled to yield the final product sevelamer hydrochloride. The presence of the allylamine is not detected or less than 1 ppm in the final product sevelamer hydrochloride.

While the invention has been described and exemplified in sufficient detail for those skilled in this art to make and use it, various alternatives, modifications, and improvements should be apparent without departing from the spirit and scope of the invention. One skilled in the art readily appreciates that the present invention is well adapted to carry out the objects and obtain the ends and advantages mentioned, as well as those inherent therein. Modifications therein and other uses will occur to those skilled in the art. These modifications are encompassed within the spirit of the invention and are defined by the scope of the claims.

What is claimed is:

1. A membrane filtration system for separating polymeric liquid with different molecular weights comprising:
(a) one or more membrane filtration units, wherein the membrane filtration unit compiled in a pressure vessel;
(b) a feeding pipe coupled to the membrane filtration unit, wherein the feeding pipe with one end fixed at one end of the pressure vessel;
(c) a concentrate exit pipe coupled to the membrane filtration unit, wherein the concentrate exit pipe with one end fixed at one end of the pressure vessel, and the other end of the concentrate exit pipe coupled to the feeding pipe by a succeeding unit;
(d) a filtrate tank surrounded by the membrane filtration unit(s), which for collecting a filtrate liquid that flows from the pressure vessel;
(e) a filtrate exit pipe connected to said filtrate tank, which involves removing the filtrate liquid from the filtrate tank; and
(f) a pump system coupled to the membrane filtration unit, which provides a pressure and forces a feed liquid against the membrane filtration unit.

2. The system of claim 1, wherein the membrane filtration unit containing two or more membrane filter elements.

3. The system of claim 2, wherein the membrane filter element is a semi-permeable membrane having a nominal pore size.

4. The system of claim 1, wherein the filtrate liquid is polymeric liquid with a low average molecular weight.

5. The system of claim 1, wherein the pump system enables the membrane filtration system in a continuous motion by directing a concentrate liquid back to the feeding pipe.

6. A method for preparing poly(allylamine) hydrochloride comprising the steps of:
(a) reacting an aqueous of allylamine hydrochloride with a polymerization agent to produce a crude poly(allylamine) hydrochloride; and
(b) performing the crude poly(allylamine) hydrochloride to prepare poly(allylamine) hydrochloride with an average molecular weight of over 2,000 daltons by using a membrane filtration system.

7. The method of claim 6, wherein the polymerization agent is 2,2′-azobis(2-methylpropionamidine)hydrochloride (AAPH).

8. The method of claim 6, wherein the crude poly(allylamine) hydrochloride is performed by neutralization and distillation before step (b).

9. The method of claim 6, wherein the crude poly(allylamine) hydrochloride with not-detectable or less than 100 ppm of allylamine content.

10. A process for producing a dried sevelamer hydrochloride comprising the steps of:
(a) providing a wet sevelamer hydrochloride;
(b) performing the washing procedure with water to achieve good quality of wet sevelamer hydrochloride;
(c) compressing the wet sevelamer hydrochloride with compressing process; and
(d) drying the compressed sevelamer hydrochloride.

11. The process of claim 10, which further comprises milling the dried sevelamer hydrochloride to produce sevelamer hydrochloride powder.

12. The process of claim 11, wherein the powder of sevelamer hydrochloride has a specific gravity of greater than 1.22 g/cm³.

13. The process of claim 12, wherein the powder of sevelamer hydrochloride has a specific gravity of 1.240 g/cm³.

14. The process of claim 11, wherein the powder of sevelamer hydrochloride with not-detectable or less than 1 ppm of allylamine content.

15. The process of claim 10, wherein the wet sevelamer hydrochloride is prepared from reacting poly(allylamine) hydrochloride with a crosslinking agent in an alkaline solution.

16. The process of claim 15, wherein the crosslinking agent is epichlorohydrin (ECH), 1,4 butanedioldiglycidyl ether, 1,2 ethanedioldiglycidyl ether, 1,3-dichloropropane, 1,2-dichloroethane, 1,3-dibromopropane, 1,2-dibromoethane, succinyl dichloride, dimethylsuccinate, toluene diisocyanate, acryloyl chloride, or pyromellitic dianhydride.

17. The process of claim 15, wherein the poly(allylamine) hydrochloride is prepared from a crude poly(allylamine) hydrochloride by using a membrane filtration system.

18. The process of claim 17, wherein the crude poly(allylamine) hydrochloride is performed by neutralization and distillation before the crosslinking reaction with ECH.

19. The process of claim 17, wherein the crude poly(allylamine) hydrochloride with not more than 100 ppm of allylamine content.

20. The process of claim 10, wherein the compressing process is performed by a roller compactor.

21. A process for producing a dried sevelamer carbonate comprising the steps of:
(a) providing a crosslinked poly(allylamine) hydrochloride;
(b) neutralizing the crosslinked poly(allylamine) polymer with metal carbonate or carbon dioxide to produce a wet sevelamer carbonate;
(c) performing the washing procedure with water to achieve good quality of wet sevelamer carbonate;
(d) compressing the wet sevelamer carbonate with compressing process; and;
(e) drying the compressed sevelamer carbonate.

22. The process of claim 21, which further comprises milling the dried sevelamer carbonate to produce sevelamer carbonate powder.

23. The process of claim 22, wherein the powder of sevelamer carbonate has a specific gravity of greater than 1.240 g/cm³.

24. The process of claim 21, wherein the powder of sevelamer carbonate with not-detectable or less than 1 ppm of allylamine content.

25. The process of claim 21, wherein the neutralizing process is performed by adding potassium carbonate for anion exchange.

26. The process of claim 21, wherein the crosslinked poly(allylamine) polymer is prepared from reacting poly(allylamine) hydrochloride with a crosslinking agent in an alkaline solution.

27. The process of claim 26, wherein the poly(allylamine) hydrochloride is prepared from a crude poly(allylamine) hydrochloride by using a membrane filtration system.

28. The process of claim 27, wherein the crude poly(allylamine) hydrochloride is performed by neutralization and distillation before the crosslinking reaction with ECH to give the hydrogels of polymer.

29. The process of claim 21, wherein the compressing process is performed by a roller compactor.

30. A process for producing a dried colesevelam hydrochloride comprising the steps of:
(a) providing a crosslinked poly(allylamine) hydrochloride;
(b) neutralizing the crosslinked poly(allylamine) hydrochloride with an alkaline solution;
(c) alkylating the crosslinked poly(allylamine) polymer with 1-bromodecane and (6-bromohexyl)trimethylammonium bromide (BHTAB) to produce a crude colesevelam;
(d) reacting the crude colesevelam with sodium chloride;
(e) applying the mixture from step (d) with anion exchange to produce wet colesevelam hydrochloride;
(f) performing the washing procedure with water to achieve good quality of wet colesevelam hydrochloride; and
(g) drying the wet colesevelam hydrochloride.

31. The process of claim 30, which further comprises milling the dried colesevelam hydrochloride to produce colesevelam hydrochloride powder.

32. The process of claim 31, wherein the powder of colesevelam hydrochloride has a specific gravity of greater than 1.240 g/cm³.

33. The process of claim 31, wherein the powder of colesevelam hydrochloride with not-detectable or less than 1 ppm of allylamine content.

34. The process of claim 30, wherein the crosslinked poly(allylamine) polymer is prepared from reacting poly(allylamine) hydrochloride with a crosslinking agent in an alkaline solution.

35. The process of claim 34, wherein the poly(allylamine) hydrochloride is prepared from a crude poly(allylamine) hydrochloride by using a membrane filtration system.

36. The process of claim 35 wherein the crude poly(allylamine) hydrochloride is performed by neutralization and distillation before the crosslinking reaction with ECH to give the hydrogels of polymer.
